(19) United States
(12) Patent Application Publication
(10) Pub. No.: US 2026/0317836 A1
(43) Pub. Date: Oct. 15, 2026

(54) UNIVERSAL MICROBIAL IDENTIFICATION

(71) Applicant: THE REGENTS OF THE UNIVERSITY OF MICHIGAN, Ann Arbor, MI (US)

(72) Inventors: Pallavi Bugga, Ann Arbor, MI (US); Vishwaratn Asthana, Ann Arbor, MI (US); Jeremy Scott VanEpps, Ann Arbor, MI (US); Erika Martinez-Nieves, Ann Arbor, MI (US)

(21) Appl. No.: 18/998,837

(22) PCT Filed: Jul. 28, 2023
(86) PCT No.: PCT/US2023/071182

§ 371 Date: Jan. 27, 2025

Related U.S. Application Data
(60) Provisional application No. 63/393,550, filed on Jul. 29, 2022.

(51) Int. Cl. C12Q 1/689; C12Q 1/6895

(52) U.S. Cl. CPC C12Q 1/689; C12Q 1/6895; C12Q 2600/16

(57) ABSTRACT

Provided herein are methods, compositions, kits and systems for the detection, identification and quantification of bacteria and fungi. In particular, provided herein are methods, compositions, kits and systems comprising oligonucleotide primers that hybridize to sequence regions of nucleic acids flanking ribosomal 16S, 23S, 5S, 18S, 5.8S, and 25-28S internal transcribed spacer regions (ITSs) and other conserved genes from 2 or more different bacteria or fungi, polymerase chain reaction (PCR or qPCR) amplification, high resolution melt curve analysis and amplicon size determination for microbial identification.

### CROSS-REFERENCE TO RELATED APPLICATIONS

The present application claims priority to U.S. Provisional Application Ser. No. 63/393,550 filed Jul. 29, 2022, the entirety of which is incorporated by reference herein.

### FIELD

Provided herein are methods, compositions, kits and systems for the detection, identification and quantification of bacteria and fungi. In particular, provided herein are methods, compositions, kits and systems comprising oligonucleotide primers that hybridize to sequence regions of nucleic acids flanking ribosomal 16S, 23S, 5S, 18S, 5.8S, and 25-28S internal transcribed spacer regions (ITSs) and other conserved genes from 2 or more different bacteria or fungi, polymerase chain reaction (PCR or qPCR) amplification, high resolution melt curve analysis and amplicon size determination for microbial identification.

### BACKGROUND

When a patient presents with signs and symptoms of an infection, it may be difficult for a caregiver to determine the pathogenic microbial species in a timely manner. The conventional standards for pathogen diagnosis are culture-based detection, biochemical assay identification, and antibiotic susceptibility testing (AST). However, this approach is time intensive, often taking 12-48 hours to grow the pathogen to the point where additional biochemical assays can be performed to confirm species identity and perform AST.¹ Furthermore, culturing is limited in part by sensitivity. For example, 30% of cases of sepsis are culture negative.² This is the result of both uncommon but clinically relevant organisms being unculturable, requiring strong a priori clinical suspicion and species-specific molecular assays to identify, as well as certain infections and their associated clinical compartments being difficult to reliably assay.³⁻⁶ Altogether, these issues delay diagnosis. The utility of culturing is also affected by specificity. Blood culture contaminants, for example, may produce false positives, or obscure the true pathogen leading to inappropriate treatment.^{7, 8}

In patients with sepsis, mortality increases 8% for every hour of delay in antibiotic administration.^{9, 10} As a result, broad-spectrum antibiotics are often prescribed pending the results of various body-fluid cultures. Antibiotic resistance is one the greatest threats to global health. The Center for Disease Control (CDC) estimates that within the US alone, approximately 2.8 million infections and more than 35,000 deaths occur annually as a result of drug-resistant bacteria.¹¹ By 2050, 10 million deaths are expected worldwide annually from multi-drug resistant organisms.¹² Numerous studies have shown that the indiscriminate administration of broad-spectrum antibiotics is one of the main contributors to the increasing prevalence of antibiotic resistance.^{13, 14} As a result, treatment options are narrowing as antibiotic resistance forces physicians to resort to third- or fourth-line antibiotics, paving the way for the emergence of “superbugs” that are effectively resistant to all known antibiotics.¹⁵ In addition, broad-spectrum antibiotics may have serious side effects, including disruption of the endogenous microbiome, development of opportunistic infections, and organ toxicity.¹⁶ Broad-spectrum antibiotics are empirically given, such that the clinician is making a guess at the potential underlying organism that leaves the potential for under or overtreatment of a critically ill patient. In view of these concerns, multiple clinical practice guidelines, including those provided by the CDC, now recommend rapid de-escalation to targeted antibiotics as soon as the causative pathogen is identified.¹⁷

Conventional pathogen detection methods are limited by multiple constraints. For example, targeted and multiplex polymerase chain reaction technologies rely on species-specific primers, and are biased toward a limited number of pre-selected organisms. Variations in amplification efficiency between each target and primer set, as well as the potential for primer dimer formation between different primer sets, limit the total number of organisms which can be interrogated. Nucleic acid next-generation sequencing (NGS) is slow (requiring at least 12-24 hours, but often more for diverse samples) and costly, with a high false positive rate, due to NGS' ability to detect low-level contaminants and non-pathogenic bacterial DNA. While also able to detect low-level contaminants, the present invention provides better quantitative read-outs, to help clinicians differentiate between pathogenic and non-pathogenic bacteria (including contaminants), unlike NGS. Current nucleic acid sequencing technologies have shown minimal clinical utility, in part for this reason. Mass spectrometry, e.g., matrix-assisted laser desorption/ionization-time of flight (MALDI-TOF) is slow (e.g., 24-120 hours), costly, labor intensive, reliant on complex technology, and sensitive to small variations in sample preparation and handling. Accordingly, new methods, compositions, systems and kits are needed for improved diagnosis and management of microbial infections. A rapid (1-2 hours), culture-free, low cost, universal microbial identification system that is unbiased by pre-defined target organisms facilitates the transition from broad-to narrow- and targeted-spectrum antibiotics, and/or obviates the need for broad-spectrum antibiotics.

### SUMMARY

Provided herein are methods, compositions, kits and systems for the detection, identification and quantification of bacteria and fungi. In particular, provided herein are methods, compositions, kits and systems comprising oligonucleotide primers that hybridize to sequence regions of nucleic acids flanking ribosomal 16S, 23S, 5S, 18S, 5.8S, and 25-28S internal transcribed spacer regions (ITSs) and other conserved genes from 2 or more different bacteria or fungi, polymerase chain reaction (PCR or qPCR) amplification, high resolution melt curve analysis and amplicon size determination for microbial identification.

In some embodiments, the present invention provides a method of identifying multiple different microbes, comprising contacting nucleic acid from the multiple different microbes with two or more oligonucleotide primers that hybridize to sequence regions of the nucleic acid from the multiple different microbes that are conserved among different microbes, wherein the conserved regions flank variable ribosomal internal transcribed regions (ITS) and conserved genes to produce one or more amplification products, determining the fragment lengths of the one or more amplification products, and identifying the multiple different microbes by comparing the fragment lengths to a database of fragment lengths from a plurality of different microbes. In some embodiments, the multiple microbes are bacteria and the variable ribosomal internal transcribed regions are 16S-23S and 23S-5S variable ribosomal internal transcribed regions. In some embodiments, the multiple microbes are fungi and the variable ribosomal internal transcribed regions are 18S-5.8S and 5.8S-28S variable ribosomal internal transcribed regions.

In some embodiments, at least one of the one or more amplification products is amplified by polymerase chain reaction (PCR), quantitative PCR (qPCR) and/or colony PCR. In some embodiments, high-resolution melt curve analysis is performed on one or more amplification products. In some embodiments, the number of ITS repeats are quantified by qPCR or electrophoresis. In some embodiments, the fragment length of at least one of the one or more amplification products is determined by gel electrophoresis or capillary electrophoresis.

In some embodiments, at least one of said two or more primers comprises a mixed base and/or an inosine. In some embodiments, periodic removal of dominant amplification products is followed by replenishment of PCR reagents and serial amplification. In some embodiments, a concentration of at least one of the one or more amplification products is determined by fluorescent quantification. In some embodiments, the conserved sequence regions flank one or more variable regions in tRNA synthetase, nirS, rpo, COX1, rbcL, LSU, fus, ileS, lep, leu, pyrG, rps, dna, rnp, rpm, gyr, rec, rpl, and tuf.

In some embodiments, the present invention provides a system, comprising two or more amplification primers that hybridize to sequence regions of nucleic acid from multiple different microbes that are conserved among different microbes, wherein the conserved regions flank variable ribosomal internal transcribed regions (ITS) and conserved genes to produce one or more amplification products, one or more PCR amplification reagents, a PCR amplification instrument, a melt curve instrument, one or more capillary gel electrophoresis reagents and a capillary gel electrophoresis instrument. In some embodiments, the multiple microbes are bacteria and the variable ribosomal internal transcribed regions are 16S-23S and 23S-5S variable ribosomal internal transcribed regions. In some embodiments, the multiple microbes are fungi and the variable ribosomal internal transcribed regions are 18S-5.8S and 5.8S-28S variable ribosomal internal transcribed regions. In some embodiments, the system comprises a database of amplification product fragment lengths.

### DESCRIPTION OF THE FIGURES

FIG. 1 shows an overview of the universal microbial identification system. Universal primers targeting the flanks of conserved bacterial genes (including the 16s, 23s, and 5s ribosomal segments) are used to PCR amplify the heterogenous internal transcribed spacer (ITS) regions producing a unique amplicon signature based on the length of the ITS gaps. (a) 3 hypothetical bacterial genomes (A, B, & C) with different ITS gap lengths, the resulting (b) amplicon lengths and (c) gel electrophoretic readout that demonstrates unique identifying signatures. a) shows 3 hypothetical bacterial genomes (A, B, and C). b) shows different ITS gap lengths in the 3 bacterial genomes. C) shows a gel electrophoresis with unique identifying signatures for A, B and C bacterial genome. Additional universal primers that amplify other ITS gaps (i.e., 23s-5s region) support accuracy and resolution.

FIG. 2 shows quantification of bacterial DNA concentration using PCR cycle counting. 75 ng of S. aureus genomic DNA was PCR amplified. After every 3 cycles the reaction was paused and 2 μL of the PCR reactant was sampled and analyzed by PAGE, after which the PCR reaction was resumed. This process was repeated until cycle 35. In parallel, an identical reaction was carried out using SYBR Green qPCR, albeit without pausing the reaction and aliquoting out reactant. a) Unique bands corresponding to S. aureus emerge at cycle 21 of the PCR reaction. b) Plotting the band intensity vs cycle number shows the characteristic log 2-linear relationship that is shared with the fluorescence vs cycle number for the same reaction run using qPCR. The Ct value for that sample was 21.

FIG. 3 shows sensitivity and specificity of the universal bacterial identification system. a) To assess sensitivity, serial 10× dilutions of E. coli genomic DNA (75 ng) were PCR amplified for 60 cycles and visualized by PAGE. Each dilution was run in a separate lane. b) To assess specificity, human genomic DNA, E. coli genomic DNA, or a combination of the two were PCR amplified and visualized by PAGE. Lane 1: ladder; Lane 2: water; Lane 3: human DNA only, 35 cycles; Lane 4: human DNA only, 60 cycles; Lane 5: E. coli DNA only, 35 cycles; Lane 6: water; Lane 7:1:1 human: E. coli DNA ratio, 60 cycles; Lane 8:10:1 human: E. coli DNA ratio, 60 cycles; Lane 9:100:1 human: E. coli DNA ratio 60 cycles; Lane 10:1,000:1 human: E. coli DNA ratio, 60 cycles; Lane 11:10,000:1 human: E. coli DNA ratio, 60 cycles.

FIG. 4 shows amplification of diverse co-mixtures of bacteria in a single reaction. Varying ratios of E. coli to B. subtilis were PCR amplified for 35 cycles and visualized by polyacrylamide gel electrophoresis (PAGE). At an E. coli to B. subtilis ratio of 1:1, 75 ng of E. coli genomic DNA was mixed with 75 ng of B. subtilis genomic DNA. At 1:10, 7.5 ng of E. coli genomic DNA was mixed with 75 ng of B. subtilis DNA etc. Both species are readily identified when present within an order of magnitude of each other.

FIG. 5 shows simulated amplification profiles comprising amplicon length signatures for 45 of the most common clinical pathogens using 16S-23S universal primer sets. The universal primers used by the universal microbial diagnostic system of the present invention generate a unique amplicon signature for each pathogen.

FIG. 6 shows simulated amplification profiles comprising expected amplicon length signatures for 45 of the most common clinical pathogens using the 23s-5s universal primer sets. The universal primers used by the universal microbial diagnostic system of the present invention generate a unique amplicon signature for each pathogen.

FIG. 7 shows simulated amplification profiles with associated ITS repeats comprising expected amplicon length signatures and associated ITS repeats for 45 of the most common clinical pathogens using the 23S-5S universal primer sets.

FIG. 8 shows simulated amplification profiles and expected amplicon length signatures for 188 clinically infectious pathogens using the 16S-23S universal primer set in red and the 23S-5S universal primer set in green. Two primers in combination generate a unique amplicon signature for 97 of 188 pathogens in the database. The remaining pathogens with overlapping amplicon signatures are distinguished using high-resolution melt curves and ITS ratios.

FIG. 9 shows experimental amplicon signatures generated using universal ITS primers. a) PAGE of PCR reactants from DNA isolated from different bacterial species. b) analysis results of the gel. Band analysis was restricted to PCR products <1,000 bp which are unlikely to be spurious. Each PCR reactant profile is unique for each species.

FIG. 10 shows a molecular weight (MW) calibration curve. For the gel in FIG. 9, the molecular weights of the ladder are plotted against the relative migration distance in the gel. An exponential fit to the data is shown. A unique curve is generated for each gel to account for unique differences in gel migration.

FIG. 11 shows high-resolution melt data. a) Six bacteria with overlapping amplicon signatures after amplification with the 16S-23S and 23S-5S universal primer sets generate unique melt curves in silico as seen via uMELT. The unique melt signatures distinguish the 6 bacteria based on variances in sequence composition. b) Standard melt curve analysis was experimentally performed on 6 representative bacteria i.e., B. subtilis, C. jejuni, E. coli, K. pneumoniae, P. aeruginosa, and S. aureus.

FIG. 12 shows assay performance in fungal samples. Candida albicans was amplified using a universal fungal primer pair designed against the 18S-5.8S ITS region. Varying concentrations of the amplified fungal DNA ranging from 10 to 989 ng/uL were analyzed via electrophoresis on a 10% PAGE gel.

FIG. 13 shows simulated amplification profiles with corresponding ITS repeats comprising expected amplicon length signatures and associated ITS repeats for 45 of the most common clinical pathogens using the 16S-23S universal primer sets.

FIG. 14 shows simulated amplification profiles with corresponding ITS repeats. Expected amplicon length signatures and associated ITS repeats were generated for 188 clinical pathogens using the 16S-23S universal primer set in red and the 23S-5S universal primer set in green. The number of repeats ranges from 0-20.

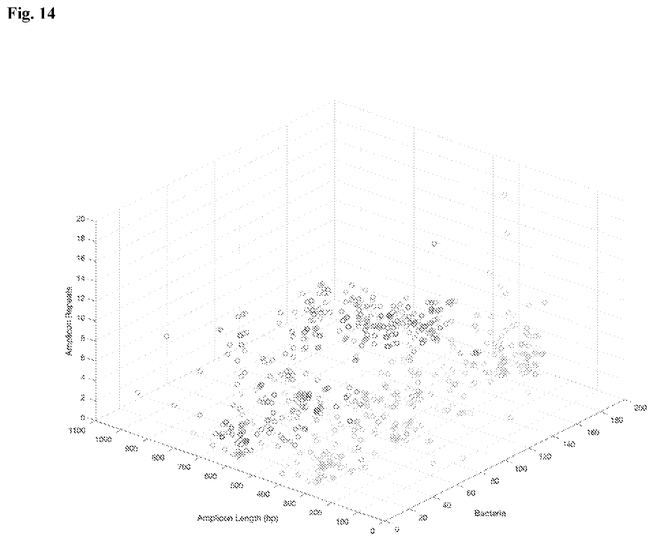

FIG. 15 shows assay performance in clinical samples, a) PAGE. b) qPCR results of serial 10× dilutions of E. coli in porcine urine amplified using a universal primer set of the present invention. Error bars show the standard errors of 4 independent samples with triplicate measures. c) PAGE results of serial 10× dilutions of E. coli in human blood. d) PAGE results of human clinical urine samples with laboratory confirmed urinary tract infections (sample 1=E. coli, sample 2 & 3=P. aeruginosa).

### DEFINITIONS

To facilitate an understanding of the present disclosure, a number of terms and phrases are defined below. While the invention will be described in conjunction with certain representative embodiments, it will be understood that the invention is not limited to these illustrative examples. One skilled in the art will recognize many methods and materials similar or equivalent to those described herein may be used in the practice of the present invention. The present invention is in no way limited to the methods and materials described.

Unless defined otherwise, technical and scientific terms used herein have the meaning commonly understood by one of ordinary skill in the art to which this invention belongs. Although any methods, devices, and materials similar or equivalent to those described herein can be used in the practice of the invention, certain methods, devices, and materials are described herein. It is further to be understood that all base sizes or amino acid sizes, and all molecular weight or molecular mass values, given for nucleic acids or polypeptides are approximate, and are provided for description.

As used in this disclosure, including the appended claims, the singular forms “a,” “an,” and “the” include plural references, unless the content clearly dictates otherwise, and are used interchangeably with “at least one” and “one or more.”

As used herein, the term “about” represents an insignificant modification or variation of the numerical value such that the basic function of the item to which the numerical value relates is unchanged.

As used herein, “protein” is used synonymously with “peptide,” “polypeptide,” or “peptide fragment.” A “purified” polypeptide, protein, peptide, or peptide fragment is substantially free of cellular material or other contaminating proteins from the cell, tissue, or cell-free source from which the amino acid sequence is obtained, or substantially free from chemical precursors or other chemicals when chemically synthesized.

As used herein, “modulate” means to alter, either by increasing or decreasing, the activity of a gene or protein. The term “inhibit”, as used herein, means to prevent or reduce the activity of gene or protein.

As used herein, the term “bioactivity” indicates an effect on one or more cellular or extracellular process (e.g., via binding, signaling, etc.) that can impact physiological or pathophysiological processes. Ranges provided herein are understood to be shorthand for all of the values within the range. For example, a range of 1 to 50 is understood to include any number, combination of numbers, or sub-range from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise). Any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used herein, the term “in vitro” refers to an artificial environment and to processes or reactions that occur within an artificial environment. In vitro environments can consist of, but are not limited to, test tubes and cell lysate. The term “in vivo” refers to the natural environment (e.g., an animal or a cell) and to processes or reaction that occur within a natural environment.

As used herein, “universal” means not specific to a single microbial species or strain. For example, a universal primer is able to bind to and amplify multiple bacterial species and strains with equivalent amplification efficiency, and/or is able to simultaneously amplify 2 or more bacterial targets in a sample.

As used herein, a “mixed base” refers to a variable base within an oligonucleotide. If, for example, an oligonucleotide contains an A: C mixed base in a given position, 50% of oligonucleotides would contain an adenine, while the remaining 50% of oligonucleotides would contain a cytosine at that same position. The inclusion of mixed bases allows for primers to better accommodate template sequence mismatches.

### DETAILED DESCRIPTION OF THE DISCLOSURE

Provided herein are methods, compositions, kits and systems for the detection, identification and quantification of bacteria and fungi. In particular, provided herein are methods, compositions, kits and systems comprising oligonucleotide primers that hybridize to sequence regions of nucleic acids flanking ribosomal 16S, 23S, 5S, 18S, 5.8S, and 25-28S internal transcribed spacer regions (ITSs) and other conserved genes from 2 or more different bacteria or fungi, polymerase chain reaction (PCR or qPCR) amplification, high resolution melt curve analysis and amplicon size determination for microbial identification.

Antimicrobial resistance is a significant international problem. The CDC estimates that within the US alone, approximately 2.8 million infections and more than 35,000 deaths occur annually as a result of drug-resistant bacteria. Numerous studies have shown that the indiscriminate administration of broad-spectrum antibiotics is one of the main contributors to the increasing prevalence of antibiotic resistance. Culture, the conventional standard for bacterial and fungal identification is a time intensive process, often taking 24-72 hours. Further, many organisms are unculturable, requiring unique species-specific molecular assays to identify. Due to this extended diagnostic period, broad-spectrum antibiotics and other antimicrobials are prescribed to prevent worsening of the patient's condition.

Conventional pathogen detection methods and technologies are limited by multiple constraints. Multiplex PCR refers to a process by which multiple primers are added into 1 reaction mixture to simultaneously amplify one or more pathogen templates. Multiplex PCR has significant drawbacks. For example, when multiple primers are added into a shared reaction, specific primers may have differential binding affinity to the template DNA and thus differential amplification efficacy than others. As a result, species within the sample amplify at differential rates than others, making quantification and assessment of polymicrobial samples difficult or impossible. Additionally, the total number of species that can be interrogated is limited, as the likelihood of non-specific binding and primer-dimer formation increases.

Methods such as MALDI-TOF may require culturing which is often the rate limiting factor in pathogen identification. (Tran, A, Alby, K, Kerr, A, Jones, M, and Gilligan, PH (2015). Cost Savings Realized by Implementation of Routine Microbiological Identification by Matrix-Assisted Laser Desorption Ionization-Time of Flight Mass Spectrometry. J Clin Microbiol 53:2473-2479.) Culture-independent methods, such as metagenomic Next Generation Sequencing (mNGS), do not require culturing but are expensive, often costing $100-$1,000 per sample if not significantly more, and also time intensive, with a turnaround time of 1-5 days. (Gu, W, Miller, S, and Chiu, CY (2019). Clinical Metagenomic Next-Generation Sequencing for Pathogen Detection. Annu Rev Pathol 14:319-338.; Votintseva, AA, Bradley, P, Pankhurst, L, Del Ojo Elias, C, Loose, M, Nilgiriwala, K, et al. (2017). Same-Day Diagnostic and Surveillance Data for Tuberculosis via Whole-Genome Sequencing of Direct Respiratory Samples. J Clin Microbiol 55: 1285-1298; Sabat, AJ, van Zanten, E, Akkerboom, V, Wisselink, G, van Slochteren, K, de Boer, R F, et al. (2017). Targeted next-generation sequencing of the 16S-23S rRNA region for culture-independent bacterial identification—increased discrimination of closely related species. Sci Rep 7:3434.) Conventional PCR methods require a priori knowledge of what the infectious organism might be in order to perform the correct test. (Franchetti, L, Schumann, DM, Tamm, M, Jahn, K, and Stolz, D (2020). Multiplex bacterial polymerase chain reaction in a cohort of patients with pleural effusion. BMC Infect Dis 20:99; Mahony, JB, Blackhouse, G, Babwah, J, Smieja, M, Buracond, S, Chong, S, et al. (2009). Cost analysis of multiplex PCR testing for diagnosing respiratory virus infections. J Clin Microbiol 47:2812-2817.) The need for these technologies arises from the limitations of bacterial cell culture, the current standard for pathogen detection. (Giuliano, C, Patel, C R, and Kale-Pradhan, P B (2019). A Guide to Bacterial Culture Identification And Results Interpretation. P T 44:192-200.)

Other unresolved, complex issues have delayed introduction of pathogen detection and identification by DNA sequencing including next-generation sequencing. For example, a recent multicenter retrospective cohort study assessing the clinical impact of Karius diagnostic's mNGS system found little to no clinical utility of the diagnostic in large part because of the turnaround time to obtain results. (Hogan, C A, Yang, S, Garner, O B, Green, D A, Gomez, C A, Dien Bard, J, et al. (2021). Clinical Impact of Metagenomic Next-Generation Sequencing of Plasma Cell-Free DNA for the Diagnosis of Infectious Diseases: A Multicenter Retrospective Cohort Study. Clin Infect Dis. 72 (2): 239-245.) Environmental and normal flora contaminants make it difficult to assess whether a species is a clinically significant pathogen or a common colonizer and/or contaminant. As well, sequencing costs for pathogen detection and identification are substantial. MinION and Nanopore are amplification-free sequencing platforms that are more rapid than Illumina but with lower throughput. (Wang, Q, Boenigk, S, Boehm, V, Gehring, NH, Altmueller, J, and Dieterich, C (2021). Single cell transcriptome sequencing on the Nanopore platform with ScNapBar. RNA. 27 (7): 763-770.) In addition, MinION and Nanopore suffer from a number of unique limitations, including low accuracy—75% and 90% for MinION and Nanopore, respectively. (Chandak, S, Neu, J, Tatwawadi, K, Mardia, J, Lau, B, Kubit, M, et al. (2020). Overcoming High Nanopore Basecaller Error Rates for DNA Storage via Basecaller-Decoder Integration and Convolutional Codes. ICASSP 2020-2020 IEEE International Conference on Acoustics, Speech and Signal Processing (ICASSP). pp 8822-8826; Laver, T, Harrison, J, O'Neill, PA, Moore, K, Farbos, A, Paszkiewicz, K, et al. (2015). Assessing the performance of the Oxford Nanopore Technologies MinION. Biomolecular Detection and Quantification 3:1-8.) As well, the lower throughput of these technologies complicates analysis of the clinical sample because most of the sample may be dominated by human genomic DNA (gDNA) and/or endogenous flora, thereby requiring a greater number of reads/runs that increases time to end result needed to reliably identify the microbial signature of an infectious pathogen. While removal of human gDNA is an option, such methods alter the concentration of species within microbial communities. (Douglas, CA, Ivey, KL, Papanicolas, LE, Best, KP, Muhlhausler, B S, and Rogers, GB (2020). DNA extraction approaches substantially influence the assessment of the human breast milk microbiome. Sci Rep 10:123.) Moreover, sequencing-based pathogen identification technologies and platforms are costly and dependent on significant operator time.

To overcome the deficits of culture-based methods and single species molecular assays, we have developed methods, compositions, systems and kits directed to universal microbial identification. The platform provides a set of universal polymerase chain reaction (PCR) primer pairs, that target various internal transcribed spacer (ITS) regions between conserved bacterial and fungal genes, as well as other conserved genes generally, creating a distinguishable amplicon signature for different bacterial species. At least 97 commonly isolated pathogenic species are uniquely identified using this approach. These determinations have been confirmed on 7 pathogenic bacterial species, including Gram-negatives and Gram-positives, aerobes and anaerobes, and spore formers. By integrating high-resolution melt curve analysis, and quantifying the number of ITS repeats, in some embodiments the preset invention identifies up to 188 clinical pathogens. High-resolution melt curve analysis generates melt curves based on the size and sequence composition of an oligonucleotide amplicon. Melt analysis discriminates between amplicon signatures of similar length. In turn, the frequency of ITS repeats are used to further discriminate between microbial species because diverse species have differing numbers of ITS repeats present within their cells. In some embodiments, the number of the repeats are determined by first amplifying the ITS regions via qPCR, then analyzing band intensity via gel electrophoresis. Without the need for a priori knowledge of the infectious organism, the methods, compositions, systems and kits of the present invention identify the unique amplicon and melt curve signature generated by multiple bacterial and fungal species in a single reaction within 1-2 hours for substantially less cost than established diagnostic modalities. In some embodiments, the diagnostic efficacy of the platform is supported by quantification of ITS repeats. In addition to determining the identity of the infectious organism, the methods, compositions, systems and kits of the present invention further determine the concentration of each pathogen with a limit of detection of 100 colony forming units (CFU) per PCR reaction. The methods, compositions, systems and kits of the present invention are resilient to human DNA contamination at physiologic concentrations, thereby eliminating the need for complex and time intensive extraction methods, and function reliably in clinical biofluids.

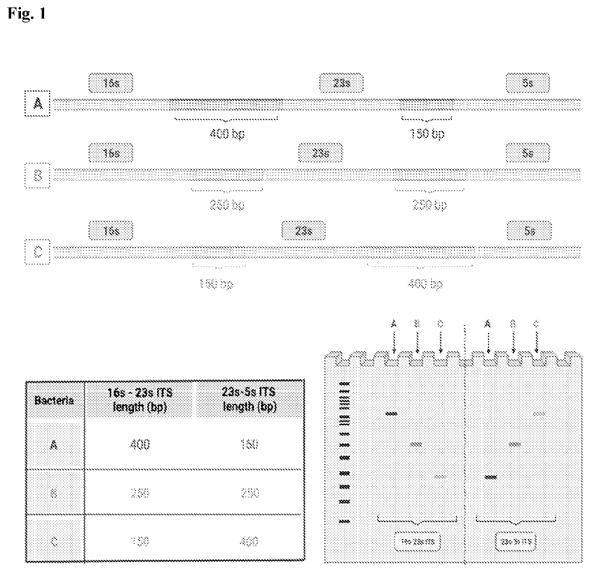

The universal microbial detection methods, systems, compositions and kits of the present invention overcome multiple limitations of conventional diagnostic technologies. In some embodiments, the present invention provides universal primers that target gaps between uniquely conserved regions in the bacterial genome, including the ribosomal 16S-23S and 23S-5S internal transcribed spacer (ITS) regions. While the 16S, 23S and 5S genes themselves are conserved with limited variation between bacteria, the ITS regions between the 3 genes comprises unique length heterogeneities that may be assessed using DNA fractionation methods such as gel electrophoresis. (FIG. 1). In some embodiments, universal primers target gaps between uniquely conserved regions in the fungal genome, including between the 18S, 5.8S, and 25-28S ITS regions. Additional ITS regions are targeted and amplified to improve the specificity.

Use of universal primers enables the methods, compositions, systems and kits to be target-agnostic such that a clinician does not require a priori knowledge of the causative pathogen to order the correct test and obtain a diagnosis, a problem commonly encountered with PCR or serology-based assays. By incorporating semiquantitative gel analysis or qPCR, the concentration of each identified organism may be determined. This information assists the clinician with discriminating a true pathogen versus contaminant/commensal organism. As well, because these primers target specific bacterial sequences, the methods, compositions, systems and kits are resilient to human genomic contamination, precluding the need for timely and costly human genomic extraction protocols. Use of robust methodologies such as PCR and electrophoresis facilitates both speed and cost, thereby making the universal bacterial identification methods, compositions, systems and kits practical clinical tools to overcome the limitations of existing diagnostic assays such as sequencing.

In some embodiments, the methods, compositions, systems and kits of the present invention comprise high-resolution melt curve analysis to distinguish differently sized amplicons to further resolve bacterial or fungal identification. This method is helpful when the size of 2 amplicons, or the combined amplicon signatures generated from the entire primer set, appear to overlap. In such cases, melt curves distinguish the 2 pathogens because Tm is more sensitive to sequence variations (e.g., GC content). In some embodiments, melt curve analysis provides assurance that methods, compositions, systems and kits of the present invention perform robustly. In some embodiments, melt curve analysis is performed in combination with qPCR.

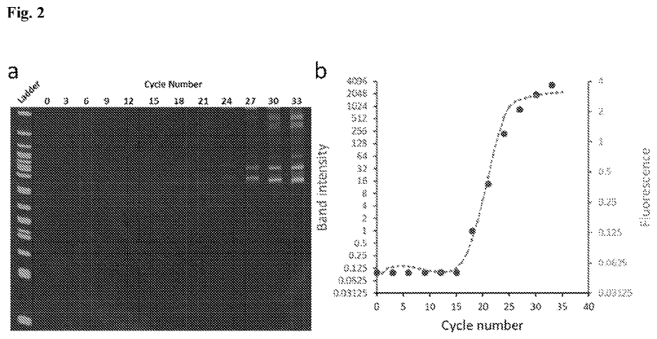

In some embodiments, the methods, compositions, systems and kits of the present invention incorporate qPCR amplification in combination with quantification of gel electrophoresis band intensity in order to determine ITS ratios. In some embodiments, the bacterial or fungal ITS region of interest is amplified by qPCR to the threshold cycle (CT), and then characterized by gel electrophoresis. In some embodiments, relative band intensities are quantified to determine ITS ratios. In some embodiments, the threshold cycle number is prospectively determined, whereupon qPCR amplification is abrogated at threshold and immediately characterized via electrophoresis. In some embodiments, a cycle counting method (FIG. 2) is used to retrospectively quantify gel band intensities at CT.

The universal bacterial identification system provided herein addresses multiple unmet needs in health care. The system identifies a bacterial or fungal pathogen within a few hours. Without having a priori knowledge of the infectious organism, the system determines both the identity and concentration of each pathogen in a clinical sample without the need for culturing, and detects as few as a single infectious cell. PCR is inexpensive with primers and reagents for each reaction costing on the order of cents (the next cheapest diagnostic modality is MALDI-TOF at approximately $10 per sample).¹⁹ The system is also resilient to human genomic contamination precluding the need for timely or costly methods to clear human DNA.

In some embodiments of the present invention, custom universal primers bind to conserved bacterial or fungal genes and amplify non-conserved gaps located in-between known as internal transcribed spacers (ITS). Because ITS regions are non-coding, and as a result not well conserved due to a lack of evolutionary pressure, these regions tend to have significant length and sequence heterogeneity between bacterial species.²⁰ Diversity in ITS length facilitates discrimination at the species level using robust and low-cost techniques including, for example, PCR and gel electrophoresis.

Simulation data of the custom universal primers against 188 of the most common clinical bacterial pathogens shows that a unique amplicon signature is generated for 97 specified bacterial species. High-resolution melt curve analysis can discriminate the remaining 91 species. Use of additional universal primers targeting flanking regions between or within other conserved bacterial genes, outside of the 16s-23s and 23s-5s ITS regions, supports the discriminatory power of the system by generating additional amplicon profiles. The ratio of ITS repeats between amplicons improves specificity (e.g., B. subtilis has 2 bands at an amplicon ratio of 1:4, while K. pneumoniae has 3 bands at an amplicon ratio of 1:4:3). The methods, systems, compositions and kits of the present invention are not limited to the exemplary 188 bacteria analyzed. The database of bacterial species against which primers are designed in silico is expandable to comprise additional genomes of interest. In the course of development of embodiments of the present invention, the capacity to generate a unique amplicon signature using universal ITS primers was confirmed in a cohort of bacteria including Gram negatives vs positives, aerobes vs anaerobes, and spore formers, and nonoverlapping readout predicted by simulation is maintained.

In some embodiments, bacterial samples, at clinically relevant concentrations (>10,000 CFU/mL) are sufficiently amplified after 35 cycles using a rapid cycling protocol that takes under an hour to complete, followed by time needed to perform gel electrophoresis, gel staining, and imaging using conventional apparatuses. Capillary electrophoretic apparatuses and microfluidic platforms reduce DNA fragmentation analysis to approximately 30 minutes. 21,22

In addition to identity, determining the concentration of the pathogenic strain is often critical. Providing a quantitative readout differentiates infectious from commensal bacteria and/or contaminants. For example, a pathogen load of <100,000 CFU/mL in urine is often considered non-infectious.²³ Similarly, low levels of circulating bacterial DNA may be found in blood which may be picked up as a false positive by endpoint assays such as multiplex PCR or Next Generation Sequencing (NGS). Accordingly, detecting bacterial DNA is not necessarily equivalent to detecting live bacteria. Free floating bacterial DNA is confined to low circulating levels because it is degraded by ubiquitously expressed DNases, so detecting bacterial DNA above background levels is informative.²⁴ In some embodiments, the present invention provides DNA precipitation, nucleases, chelation agents and/or DNA intercalators to remove DNA not sequestered within a viable membrane before PCR amplifying the sample. In some embodiments, the clinical sample is filtered after treatment with a nuclease to concentrate bacterial or fungal cells and to remove extracellular DNA, debris, or other particulate matter. In some embodiments, human cells are selectively lysed using a non-ionic detergent and treated with DNAse to remove human genomic contamination. In some embodiments, selective lysis, DNA nucleases, and filtration are used together to purify bacterial or fungal cells.

In some embodiments, concentration estimates can be obtained from qPCR amplification. In some embodiments, concentration is determined by sampling a very small volume (<1 μL) of the PCR reaction every few cycles and running the sample on a gel. From left to right, the lane in which the bands appear first indicates the relative Ct of the sample, which is converted into a concentration (CFU/mL) using an oligonucleotide standard template. In some embodiments, oligonucleotide standards are run in parallel with the same reaction matrix (to recreate the effect of potential background inhibitors and lysates that may present within the target sample). In some embodiments, the reaction is started in a qPCR apparatus and terminated once the reaction amplification has reached fluorescence threshold. At this point it is transferred to a gel while the oligonucleotide standards continue to cycle in the qPCR apparatus. Accordingly, the CT noted on qPCR aligns closely with the appearance of bands via gel electrophoresis.

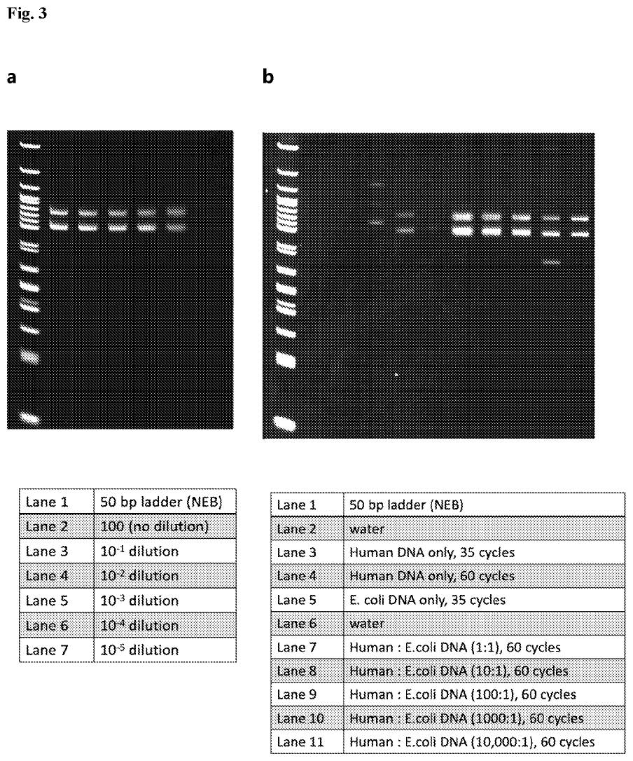

In the course of development of the present invention, we determined the sensitivity of the system using serial log dilutions of bacterial DNA. PCR is capable of identifying even a single nucleic acid strand with sufficient cycling. We found that the limit of detection (LOD) using standard bacterial DNA extraction and amplification, was approximately 250 bacteria in a PCR reaction tube. Extrapolating this to conventional units (CFU/mL), this value is equivalent to 250,000 CFU/mL at the cusp of clinical relevance in select biofluids including urine, sputum, and pus.^{20, 26, 27} FIG. 3 shows the abrupt drop off in signal as opposed to gradual fading between the 1/10,000-fold dilution and 1/100,000-fold dilution indicating that the limits of serial dilution and bacterial DNA extraction using standard experimental technique, as opposed to intrinsic limits in sensitivity of the system of the present invention, account for the loss of signal at the 1/100,000-fold dilution. Thus, the methods, compositions, systems and kits of the present invention are capable of detecting as few as a single bacterium in a reaction. In some embodiments, centrifuging and concentrating the bacteria is performed prior to DNA extraction. In some embodiments, the bacteria are concentrated by filtration. Methods of PCR amplifying bacteria in the biofluid of origin, as opposed to initial extraction of the DNA, enhance sensitivity.²⁸ In some embodiments, the methods, compositions, systems and kits of the present invention comprise colony PCR. Colony PCR is performed directly on bacterial cells without the need to separately extract and purify cellular DNA. Bacterial cells (or colonies) are lysed, and PCR reagents are added directly to the lysate. Colony PCR shortens the time from sample-to-answer, allowing for more rapid administration of tailored antibiotics, and provides less loss in DNA yield than separately extracting and purifying bacterial DNA before amplification.

While existing and emerging diagnostics, including MALDI-TOF and next-generation sequencing (NGS), are sensitive in theory, they are limited by noise that originates from human cells and genomic DNA. This is especially pronounced in blood where human cells and DNA can outnumber a bloodborne bacterial infection by several orders of magnitude.¹⁸ The custom primers of the universal bacterial identification system of the present invention and their genomic targets, i.e., the flanks of the 16s-23s and 23s-5s ITS regions, are sufficiently specific to the bacterial kingdom such that off target binding and amplification of the human genome is unfavorable and unlikely. We show both in silico and in vitro that human DNA, at concentrations typically found in blood, does not appreciably amplify within a typical number of PCR cycles (i.e., 35 cycles), and produces only very faint but distinct bands when over cycled (i.e., 60 cycles), suggesting that any signal generated by human DNA can be reliably deconvoluted from bacterial DNA. Even at the limit of detection of the system, human genomic DNA does not interfere with bacterial DNA amplification and detection, despite being up to 10⁵-fold in excess by mass. These features of the present invention preclude the need for timely and costly human DNA extraction methodologies that have proven to be problematic for emerging bacterial diagnostic technologies such as mass spectrometry and next-generation sequencing.

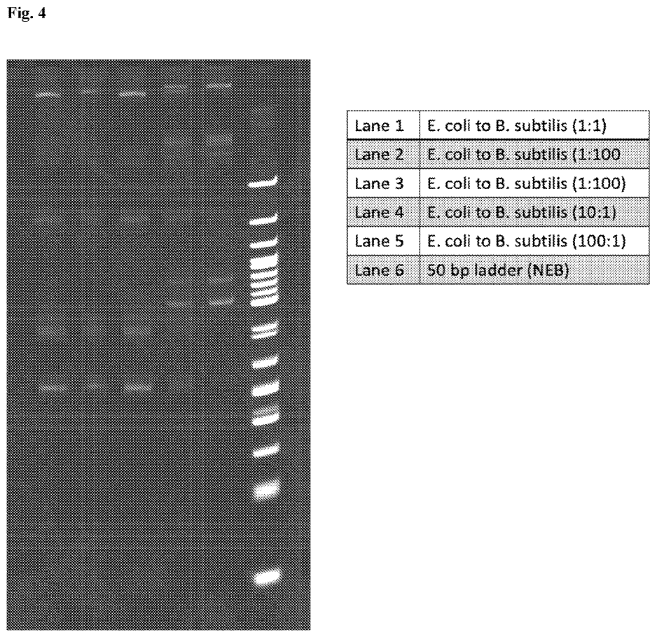

On occasion, clinical samples comprise a mixture of pathogenic bacterial strains. In these scenarios, or in circumstances in which contaminant bacteria obscure the actual pathogenic strain of interest, the ability to discern multiple bacterial species in a single reaction is helpful. In experiments conducted in the course of development of the present invention, we show that multiple bacteria may simultaneously be detected when present within an order of magnitude of each other by molarity (FIG. 4). Above this point, the more concentrated species hinders amplification of less concentrated species, likely via consumption of PCR reagents and other mechanisms.²⁵

To test the systems of the present invention in a clinical setting, we tested the universal bacterial identification system in clinical urinary tract infection (UTI) samples and observed that the system reliably identifies the culprit pathogen without the need for culturing, thereby paving the way for direct translation to clinical use. The universal bacterial identification methods, compositions, systems and kits of the present invention satisfy a longstanding clinical unmet need. At present, there is no analogous technology to the universal microbial identification system demonstrated herein. In some embodiments, the universal microbial identification system of the present invention is distinct from a multiplex platform that uses multiple species-specific primer pairs to assay an upper limit of 10 to 20 pathogens at a time.²⁹ To the contrary, in some embodiments, the universal microbial diagnostic methods, systems, compositions and kits of the present invention provide a single primer pair to interrogate the clinical pathogen database comprising hundreds of potential microbial and bacterial pathogens, and is not limited to a single clinical compartment or bacterial classifier (e.g., Gram positive vs. Gram negative) vs. conventional bioassays.³⁰ For example when an assay is confined to testing for the most common pathogens in a biofluid, there is always a risk if the test comes back negative that the causative pathogen was not included in the original test panel. As a result, clinicians are obligated to treat broadly with antibiotics to avoid a misdiagnosis and detrimental outcome. In some embodiments, the microbial pathogen is a bacterium, a fungus, or a prokaryote with ITS sequences.

The universal microbial identification systems, methods, compositions, reaction mixtures and kits of the present invention rapidly identify a pathogen within several hours. Without requiring a priori knowledge of the infectious organism, the system determines both the identity and concentration of each pathogen in a clinical sample without the need for cell culture, and may detect as few as several or a single infectious cell. The present invention provides strong advantages in cost, ease of use, operator engagement, and time consumption. The system of the present invention is resilient to human genomic contamination precluding the need for timely or costly methods to eliminate human DNA. The universal bacterial identification methods, compositions, systems and kits of the present invention provide a major resolution to the decades-long struggle to rapidly and accurately identify clinical pathogens.

In some embodiments, the present invention comprises an instrument or system in a clinical pathology laboratory. In some embodiments, the instrument or system is a point of care apparatus. In some embodiments, the instrument or system comprises a sample collection vessel into which a patient's sample is deposited. In some embodiments, the vessel is in fluid connection with an internal apparatus capable of performing colony-qPCR. In some embodiments, the instrument or system administers PCR reagents directly into the sample. In some embodiments, the instrument or system comprises a thermocycler. In some embodiments, the instrument or system quantifies fluorescence of the amplification productions. In some embodiments of the instrument or system, the sample is assessed through an internal capillary electrophoresis apparatus. The observed and measured band electrophoretic band sizes and patterns are then aligned with a band and pattern database via a CPU processer contained within the instrument or system. In some embodiments in clinical use, a patient sample is collected, the sample is directed to a suitable laboratory, facility or point of care end-user, and an instrument or system of the present invention performs colony qPCR, followed by capillary electrophoresis. The electrophoretic band sizes and patterns are then compared to a pre-generated database of band sizes and patterns to identify one or more microbial species in the sample.

In some embodiments, the methods, compositions, systems and kits of the present invention identify a microbe in a sample. In some embodiments, the sample is a biological sample or an environmental sample. In some embodiments, the biological sample is from a mammal. In some embodiments, the mammal is a human. In some embodiments, the biological sample is a fluid sample or a tissue sample.

### EXPERIMENTAL EXAMPLES

The following examples are provided to demonstrate and further illustrate certain embodiments and aspects of the present disclosure and are not to be construed as limiting the scope thereof.

### Experimental Methods

### Primer Design

Primers amplifying the 16s-23s and 23s-5s ITS regions were derived from published sources.^{31,32,33} In some embodiments of the present invention, in a first primer design step, common pathogenic bacteria and fungi are compiled in a database and aligned at their 16S, 23S, or 5S sites (for bacteria), 18S, 5.8S, 25S, or 25-28S sites (for fungi), together with other conserved regions of interest. In a second step, the greatest conserved region across microbial species is identified within the targeted sites. In a third step, candidate primer sequences are generated from regions of greatest conservation between species. In a fourth step, primers and primer pairs with the least binding affinity for human genomic DNA are selected. In a fifth step, universal primers are modified to include mixed bases (in a 1:1 ratio) as well as inosine, a universal base. Inosine is included on the 3′ end of the primer to facilitate polymerase extension in the presence of a potential mismatch near the tail end of the primer binding site. Use of additional mixed and universal bases is avoided in some embodiments to prevent off-target binding and amplification. Examples of primer sequences are shown in Table 1.

TABLE 1
Universal ITS primer sequences*

| Primer | Sequence |
|---|---|
| 16S FWD primer | AAGTCGTAACAAGGTARCCGI |
| | (SEQ. ID NO. 1) |
| 23S REV primer | YGCCAAGGCATCCACCI (SEQ. ID NO. 2) |
| 23S FWD primer | GCTGAAAGCATCTAAGI (SEQ. ID NO. 3) |
| 5S REV primer | CGTTTCACTTCTGAGTTCGGI |
| | (SEQ. ID NO. 4) |
| 16S FWD primer | CTTGTACWCACCGCCCG (SEQ. ID NO. 5) |
| 23S REV primer | TTCGYTCGCCRCTACT (SEQ. ID NO. 6) |

*R = A or G; Y = C or T; I = inosine

### Primer ITS Amplification and Simulation

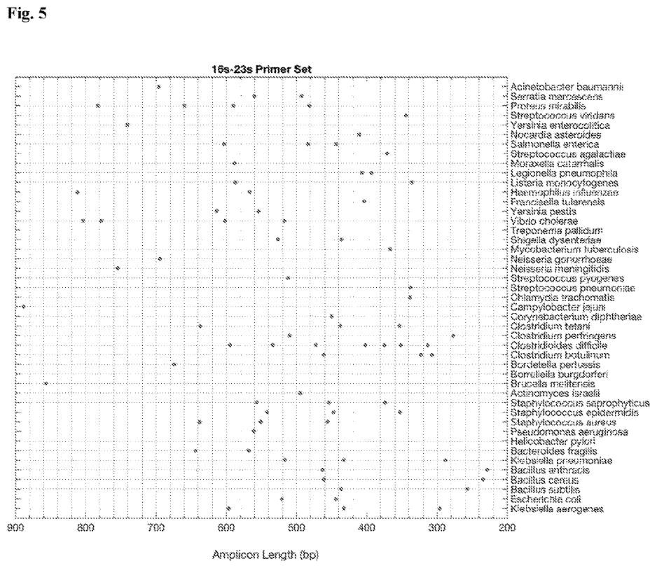

After primer design, a Matlab script was developed to establish primer binding sites and subsequent amplification profiles for each bacterial genome. The code searches each bacterial genome for primer binding sites by first probing for complementary base pair matches, followed by calculation of the binding affinity (ΔG) of each hit using integrated Nucleic Acid Package (NUPACK) code. Only primer binding sites with a ΔG<−9 kcal/mole were found to be adequately stable to form a dimer, a prerequisite for amplification. Next, each primer is elongated 5′→3′ until it reaches the next inversely oriented primer binding site along the genome, the intervening sequence of which is considered an amplicon. Only amplicons <1,000 bp are included in the identity matrix generated for each universal ITS primer pair (FIGS. 5, 6, 7, 8). This process was also performed for fungal genomes. FAST-All (FASTA) files containing bacterial genomic sequences were obtained from National Center for Biotechnology Information (NCBI). (Table 2.)

TABLE 2
Bacterial Database List

| | | |
|---|---|---|
| Gardnerella vaginalis | Clostridium tetani | Streptococcus |
| | | sobrinus |
| Micrococcus luteus | Veillonella dispar | Streptococcus suis |
| Mycobacterium | Mycoplasmosis | Streptococcus uberis |
| tuberculosis | fermentans | |
| Mycobacterium | Mycoplasmoides | Streptococcus |
| abscessus | genitalium | viridans |
| Mycobacterium avium | Malacoplasma | Staphylococcus |
| | penetrans | aureus |
| Mycobacterium | Mycoplasmoides | Staphylococcus |
| fortuitum | pneumoniae | epidermis |
| Mycobacterium | Erysipelothrix | Staphylococcus |
| kansasii | rhusiopathiae | haemolyticus |
| Mycobacterium leprae | Enterococcus avium | Staphylococcus |
| | | saprophyticus |
| Mycobacterium | Enterococcus durans | Listeria ivanovii |
| haemophilum | | |
| Mycobacterium | Enterococcus faecalis | Listeria |
| marinum | | monocytogenes |
| Mycolicibacterium | Enterococcus faecium | Listeria seeligeri |
| phlei | | |
| Mycobacterium simiae | Enterococcus | Listeria welshimeri |
| | flavescens | |
| Mycolicibacterium | Enterococcus | Bacillus anthracis |
| smegmatis | gallinarum | |
| Mycobacterium | Streptococcus | Brevibacillus brevis |
| ulcerans | acidominimus | |
| Mycobacterium xenopi | Streptococcus | Bacillus cereus |
| | agalactiae | |
| Prescottella equi | Streptococcus equinus | Lysinibacillus |
| | | fusiformis |
| Actinomyces israelii | Streptococcus canis | Bacillus mycoides |
| Actinomyces naeslundii | Streptococcus equi | Bacillus subtilis |
| Cutibacterium acnes | Streptococcus ferus | Fusobacterium |
| | | necrophorum |
| Rothia dentocariosa | Streptococcus | Fusobacterium |
| | intermedius | nucleatum |
| Nocardia asteroides | Lactococcus lactis | Streptobacillus |
| | | moniliformis |
| Nocardia nova | Streptococcus milleri | Borellia burgdorferi |
| Corynebacterium | Streptococcus mitis | Borrelia recurrentis |
| amycolatum | | |
| Corynebacterium | Streptococcus mutans | Borrelia turicatae |
| jeikeium | | |
| Corynebacterium | Streptococcus oralis | Leptospira |
| xerosis | | interrogans |
| Corynebacterium | Streptococcus | Treponema pallidum |
| diphtheriae | pneumoniae | |
| Clostridium botulinum | Streptococcus pyogenes | Treponema denticola |
| Clostridium difficile | Streptococcus ratti | Bacteroides fragilis |
| Clostridium perfringens | Streptococcus salivarius | Bacteroides |
| | | thetaiotaomicron |
| Clostridium septicum | Streptococcus sanguis | Capnocytophaga |
| | | canimorus |
| Rickettsia conorii | Vibrio | Bordetella pertussis |
| | parahaemolyticus | |
| Rickettsia prowazekii | Vibrio vulnificus | Burkholderia cepacia |
| Rickettsia rickettsi | Francisella tularensis | Burkholderia mallei |
| Rickettsia typhi | Stenotrophomonas | Burkholderia |
| | maltophila | pseudomallei |
| Achromobacter | Alteromonas | Ralstonia |
| xylosoxidans | tetraodonis | mannitolilytica |
| Eikenella corrodens | Haemophilus | Ralstonia pickettii |
| | influenzae | |
| Neisseria gonorrhoeae | Haemophilus aegyptius | Ralstonia |
| | | solanacearum |
| Neisseria meningitidis | Haemophilus ducreyi | Acinetobacter |
| | | baumannii |
| Escherichia coli | Salmonella bongori | Salmonella enterica |
| | | subsp. enterica |
| | | serovar Enteritidis |
| Pseudomonas | Pseudomonas putida | Vibrio cholerae |
| fluorescens | | |
| Salmonella | Shigella boydii | Shigella dysenteriae |
| typhimurium | | |
| Chryseobacterium | Rickettsia conorii | Vibrio |
| meningosepticum | | parahaemolyticus |
| Porphyromonas | Rickettsia prowazekii | Vibrio vulnificus |
| gingivalis | | |
| Prevotella | Rickettsia rickettsi | Francisella tularensis |
| melaninogenica | | |
| Chlamydophila psittaci | Rickettsia typhi | Stenotrophomonas |
| | | maltophila |
| Chlamydophila | Achromobacter | Alteromonas |
| pneumoniae | xylosoxidans | tetraodonis |
| Chlamydia trachomatis | Eikenella corrodens | Haemophilus |
| | | influenzae |
| Parachlamydia | Neisseria gonorrhoeae | Haemophilus |
| acanthamoebae | | aegyptius |
| Campylobacter coli | Neisseria meningitidis | Haemophilus ducreyi |
| Campylobacter fetus | Bordetella pertussis | Haemophilus |
| | | parahaemolyticus |
| Campylobacter | Burkholderia cepacia | Haemophilus |
| hyointestinalis | | parainfluenzae |
| Campylobacter jejuni | Burkholderia mallei | Aggregatibacter |
| | | aphrophilus |
| Campylobacter lari | Burkholderia | Pasteurella |
| | pseudomallei | multocida |
| Campylobacter | Ralstonia | Citrobacter freundii |
| upsaliensis | mannitolilytica | |
| Helicobacter pylori | Ralstonia pickettii | Citrobacter koseri |
| Agrobacterium | Ralstonia solanacearum | Cronobacter |
| tumefaciens | | sakazakii |
| Bartonella bacilliformis | Acinetobacter | Edwardsiella tarda |
| | baumannii | |
| Bartonella henselae | Acinetobacteri lwoffii | Enterobacter |
| | | aerogenes |
| Bartonella quintana | Acinetobacter | Enterobacter cloacae |
| | radioresistens | |
| Brucella melitensis | Aggregatibacter | Klebsiella oxytoca |
| | actinomycetemcomitans | |
| Brucella abortus | Moraxella catarrhalis | Klebsiella |
| | | pneuomnoiae |
| Brucella canis | Acinetobacter | Morganella morganii |
| | calcoaceticus | |
| Brucella suis | Tatlockia micdadei | Pantoea agglomerans |
| Candidatus Liberibacter | Legionella longbeachae | Proteus mirabilis |
| africanus | | |
| Candidatus Liberibacter | Legionella | Proteus vulgaris |
| asiaticus | pneumophila | |
| Anaplasma | Pseudomonas | Providencia stuartii |
| phagocytophilum | aeruginosa | |
| Ehrlichia canis | Pseudomonas | Serratia marcescens |
| | alcaligenes | |
| Ehrlichia chaffeensis | Pseudomonas | Escherichia coli |
| | fluorescens | |
| Ehrlichia ruminantium | Pseudomonas putida | Salmonella bongori |
| Neorickettsia sennestsu | Vibrio cholerae | Salmonella enterica |
| | | subsp. enterica |
| | | serovar Enteritidis |
| Orientia tsutsugamushi | Vibrio mimicus | Salmonella enterica |
| | | subsp. enterica |
| | | serovar Paratyphi A |
| Haemophilus | Acinetobacteri lwoffii | Enterobacter |
| parahaemolyticus | | aerogenes |
| Haemophilus | Acinetobacter | Enterobacter cloacae |
| parainfluenzae | radioresistens | |
| Aggregatibacter | Aggregatibacter | Klebsiella oxytoca |
| aphrophilus | actinomycetemcomitans | |
| Pasteurella multocida | Moraxella catarrhalis | Klebsiella |
| | | pneuomnoiae |
| Citrobacter freundii | Acinetobacter | Morganella morganii |
| | calcoaceticus | |
| Citrobacter koseri | Tatlockia micdadei | Pantoea agglomerans |
| Cronobacter sakazakii | Legionella longbeachae | Proteus mirabilis |
| Edwardsiella tarda | Legionella | Proteus vulgaris |
| | pneumophila | |
| Salmonella enterica | Pseudomonas | Pseudomonas |
| subsp. enterica serovar | aeruginosa | alcaligenes |
| Paratyphi A | | |
| Vibrio mimicus | Providencia stuartii | Serratia marcescens |
| Shigella flexneri | Shigella sonnei | Yersinia |
| | | enterocolitica |
| Yersinia | Yersinia pestis | |
| pseudotuberculosis | | |

### Cell Culture and DNA Extraction

Bacteria obtained from American Type Culture Collection (ATCC, Manassas, VA, USA) were grown overnight in Luria-Bertani (LB) broth (purchased from Fisher Scientific, Waltham, MA, USA). (Table 3.)

TABLE 3
Bacterial strains

| Species | Strain |
|---|---|
| Campylobacter jejuni | ATCC 11168 |
| Klebsiella pneumoniae | ATCC 43816 |
| Bacillus subtilis | ATCC 6051 |
| Staphylococcus aureus | ATCC 35556 |
| Escherichia coli | ATCC 25922 |
| Pseudomonas aeruginosa | PA7 |
| Acinetobacter baumannii | BAA-1605 |

Cultures were diluted 1:30-1:50 then re-grown to an optical density of 600 nm (OD600).

DNA was extracted using the Lucigen MasterPure complete DNA & RNA purification and MasterPure gram positive DNA purification kits according to the manufacturer's instruction (Lucigen, Middleton, WI, USA). DNA was reconstituted in TE buffer and stored at −20° C. Purity and yields were quantified using a UV-Vis spectrophotometer (Nanodrop 2000, Thermo Fisher Scientific, Waltham, MA, USA). Pre-purified DNA was purchased from ATCC for Campylobacter jejuni (ATCC 11168) and Acinetobacter baumannii (BAA-1605). Candida albicans (strain: BWP17) was grown overnight (16 hr.) at 37° C. in Yeast Peptone Dextrose (YPD) media supplemented with 0.01% (m/v) uridine. Yeast DNA was extracted using the MasterPure Yeast DNA Purification Kit (Lucigen, Middleton, WI, USA). DNA was reconstituted in TE buffer and stored at −20° C. Purity and yields were quantified using a UV-Vis spectrophotometer (Nanodrop 2000, Thermo Fisher Scientific, Waltham, MA, USA).

### Universal PCR Amplification Protocol

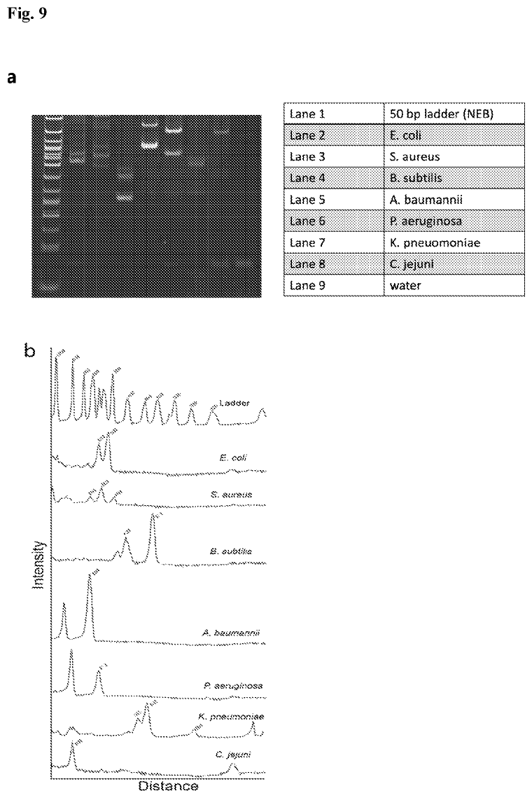

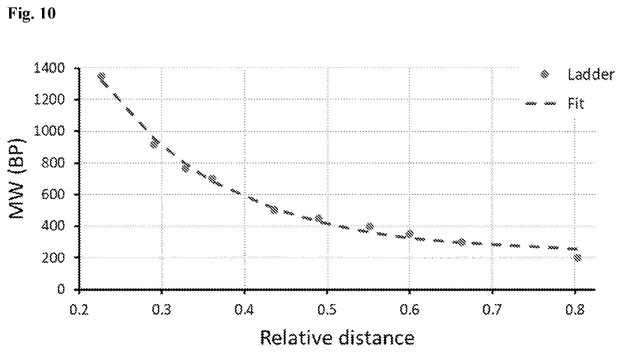

Bacterial DNA was amplified using iTaq polymerase StepOnePlus thermocycler from ThermoFisher Scientific (Waltham, MA, USA) according to the manufacturer's instruction using the following thermocycler protocol: 1) 95° C. for 3 min, 2) 95° C. for 15s, 3) 60° C. for 30s, 4) 72° C. for 1 min, and 5) Repeat steps #2 to #4 for 35 cycles. PCR reactants were subsequently separated via electrophoresis on a 10% polyacrylamide (PAGE) gel (ThermoFisher Scientific, Waltham, MA, USA) then stained with GelRed (Biotium, Fremont, CA, USA) and imaged on a ChemiDoc XRS+ molecular imager (Bio-Rad, Hercules, CA, USA). Gel images were analyzed using GelAnalyzer 19.1 (www.gelanalyzer.com). The intensity as a function of distance from the well was plotted for each lane of the gel. Peaks were identified by comparison to a standard 50 bp dsDNA ladder (New England Biolabs, Ipswich, MA, USA) that was fit to an exponential function of molecular weight versus distance. (FIG. 9 and FIG. 10)

### Quantification of Bacterial Concentration Using Cycle Counting

Staphylococcus aureus genomic DNA (150 ng) was isolated and amplified as described above. Every 3 cycles of the PCR run, the reaction was paused and 2 μL of the PCR reactant was removed, after which the PCR reaction was resumed. This process was repeated until cycle 35.

All samples were visualized by 10% PAGE with imaging and analysis as described above. In parallel, an identical reaction was carried out using SYBR green quantitative PCR (using the StepOnePlus Real-Time PCR System from Thermo Fisher Scientific, Waltham, MA, USA) according to the kit instructions, albeit without the pausing and sampling of the reactant.

### Sensitivity of the Universal Bacterial Identification System

Escherichia coli genomic DNA (150 ng) was isolated as described above. It was then serially diluted 10-fold down to a final DNA yield of 1.5 μg. Each of these dilutions were then amplified using the aforementioned PCR protocol with the caveat that the reaction was run for 60 cycles instead of 35 cycles. Results were visualized by PAGE with imaging and analysis as described above.

### Resilience to Human Contamination

Human genomic DNA (Promega, Madison, WI, USA), isolated (as described above) E. coli genomic DNA, or a combination of the two were amplified by PCR and visualized with PAGE as described previously. 375 ng (2.5 μL of 150 ng/uL) of human DNA and 75 ng (2.5 uL of 30 ng/uL) of E. coli DNA were used as PCR template. Reactions with increasing dilution ratios of E. coli to human DNA were created. Specifically, a 1:1 ratio corresponds to 2.5 μL of 150 ng/uL (375 ng) human genomic DNA and 2.5 μL of 30 ng/uL (75 ng) E. coli genomic DNA, while 1:10 corresponds to 2.5 μL of 150 ng/uL (375 ng) human genomic DNA and 2.5 μL of 3 ng/uL (7.5 ng) E. coli genomic DNA, and so on. PCR was run for either 30 or 60 cycles.

### Performance on Biofluid and Clinical Samples

E. coli was grown overnight, diluted, then re-grown to mid-log phase as described above. 50 μL of mid-log liquid culture was then added to 450 μL of porcine urine (Mohammed Tiba Laboratory, University of Michigan, Ann Arbor, MI). This process was repeated to create serial log dilutions. Concentrations (CFU/mL) of the dilutions were confirmed by plating on LB agar followed by colony enumeration. DNA was subsequently extracted, PCR amplified, and visualized by PAGE as outlined above.

For evaluation of blood samples, 3-5 mL of clinical blood samples were extracted using NaCit Vacutainer tubes. Fifty μl of mid-log E. coli culture were added to 450 ul of blood, and serial diluted as above. DNA was extracted, PCR amplified, and analyzed by PAGE.

Three urine samples from patients with confirmed urinary tract infection were obtained from the Michigan Medicine pathology laboratory. 500 μL of urine was centrifuged at 17,000 RCF for 10 min. The supernatant was discarded, and the pellet was resuspended in Gibco DPBS 1× (ThermoFisher Scientific, Waltham, MA, USA). The process was repeated twice, and followed by DNA extraction, PCR amplification, and analysis by PAGE as outlined above.

### High-Resolution Melt Curve Simulation

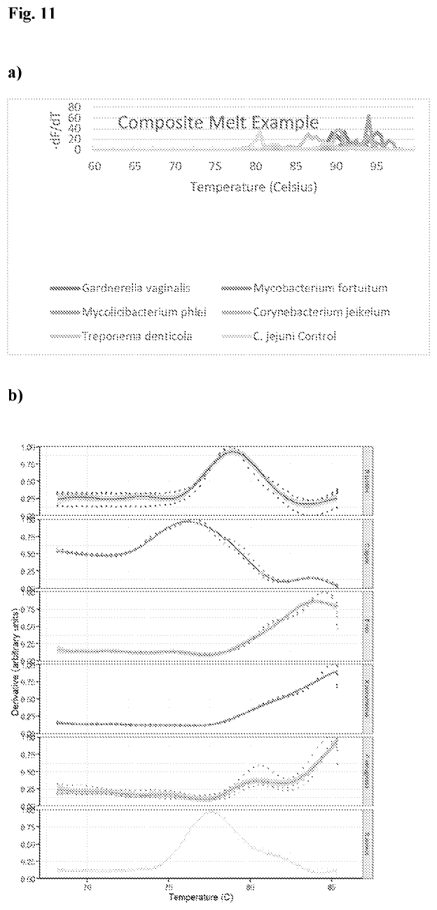

A high-resolution melt platform, uMELT, was used to perform high-resolution melt curve analysis of 188 bacteria. (Dwight, Z., Palais, R., Wittwer, C. T. uMELT: prediction of high-resolution melting curves and dynamic melting profiles of PCR products in a rich web application. Bioinformatics 27, 1019-1020, doi: 10.1093/bioinformatics/btr065 (2011). Melt curve signatures alone may be too dense and convoluted if used in isolation to distinguish and identify 188 bacteria. However, when combined with amplicon length signatures and ITS ratios, all 188 bacteria in the set are uniquely identified. For example, uMELT was performed on 6 bacteria with overlapping length signatures (G. vaginalis, M. phlei, T. denticola, M. fortuitum, C. jeikeium, and C. jejuni) to show the discrimination power of high-resolution melt curve analysis of the present invention. (FIG. 11)

### Melt Curve Analysis

Six representative bacteria (B. subtilis, C. jejuni, E. Coli, K. pneumoniae, P. aeruginosa, and S. aureus) were amplified using SYBR green quantitative PCR using the StepOnePlus Real-Time PCR System (Thermo Fisher Scientific, Waltham, MA, USA) according to kit instructions, and using the following thermocycler protocol: 1) 95° C. for 3 min, 2) 95° C. for 15s, 3) 60° C. for 30s, 4) 72° C. for 1 min, and 5) Repeat steps #2 to #4 for 35 cycles. Following amplification, melt curves were generated on the same instrument by ramping up from 60° C. to 95° C. at 0.3° C./sec. (FIG. 11)

### Performance on Fungal Samples

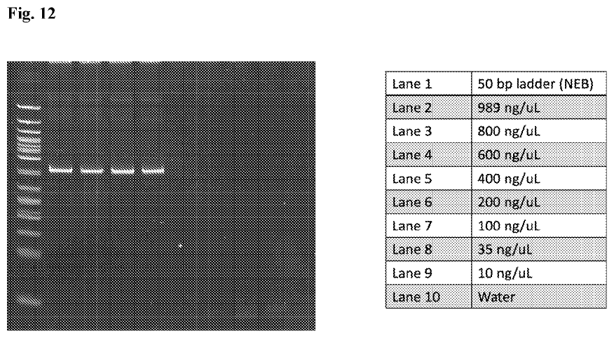

Candida albicans DNA was extracted and purified as above. Fungal DNA was amplified using SYBR green quantitative PCR (using the StepOnePlus Real-Time PCR System from Thermo Fisher Scientific, Waltham, MA, USA) according to the kit instructions, and using the following thermocycler protocol: 1) 95° C. for 3 min, 2) 95° C. for 15s, 3) 55° C. for 30 s, 4) 72° C. for 1 min, and 5) Repeat steps #2 to #4 for 35 cycles. Amplification was performed using forward and reverse primers designed against the 18S and 5.8S conserved fungal regions, respectively: FWD-5′GTCCCTGCCCTTTGTACACA-I(Inosine) 3′ and REV-5′TTTCGCTGCGTTCTTCATCG-I(Inosine) 3′. PCR reactants were subsequently separated via electrophoresis on a 10% polyacrylamide (PAGE) gel (ThermoFisher Scientific, Waltham, MA, USA) then stained with GelRed (Biotium, Fremont, CA, USA) and imaged on a ChemiDoc XRS+ molecular imager (Bio-Rad, Hercules, CA, USA). A 50 bp DNA ladder purchased from New England Biolabs (NEB, Ipswich, MA) was used for size determination. (FIG. 12)

### Example 1—Universal Primer Sets for Bacterial Pathogens

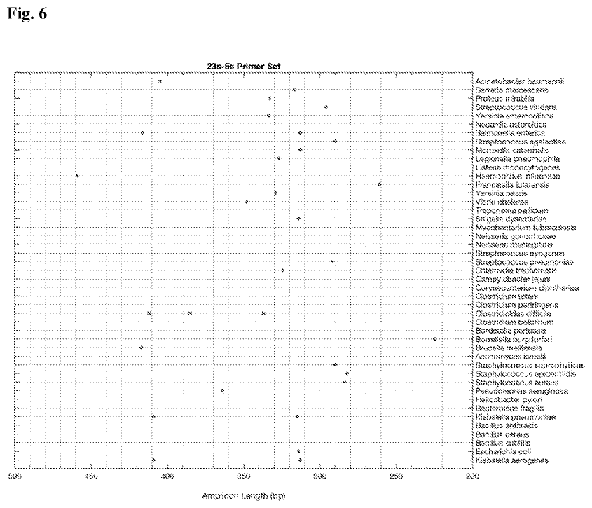

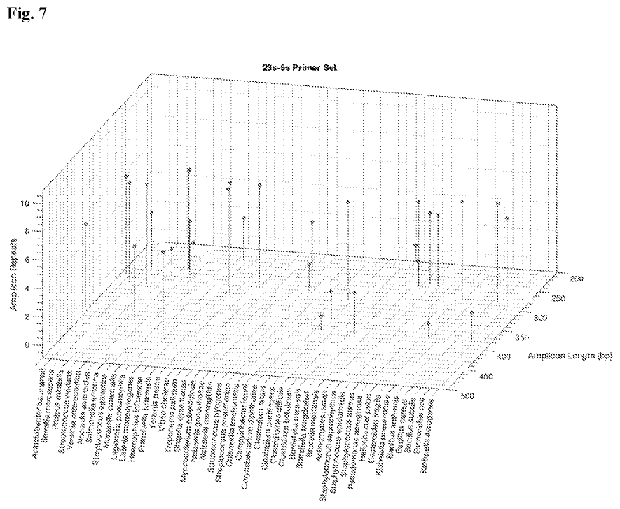

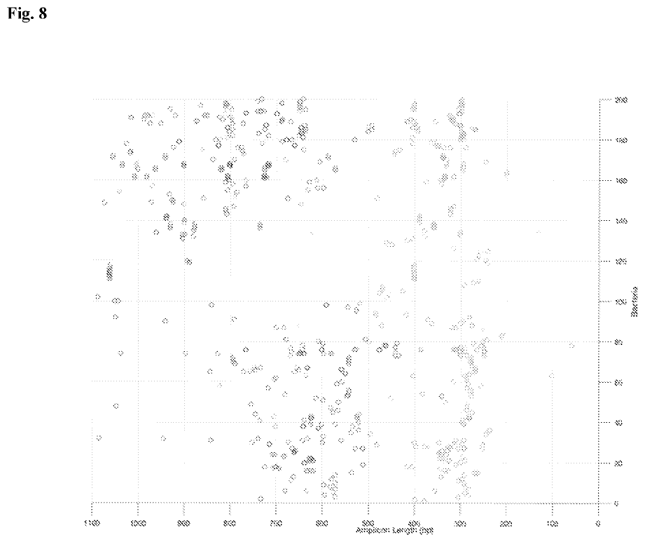

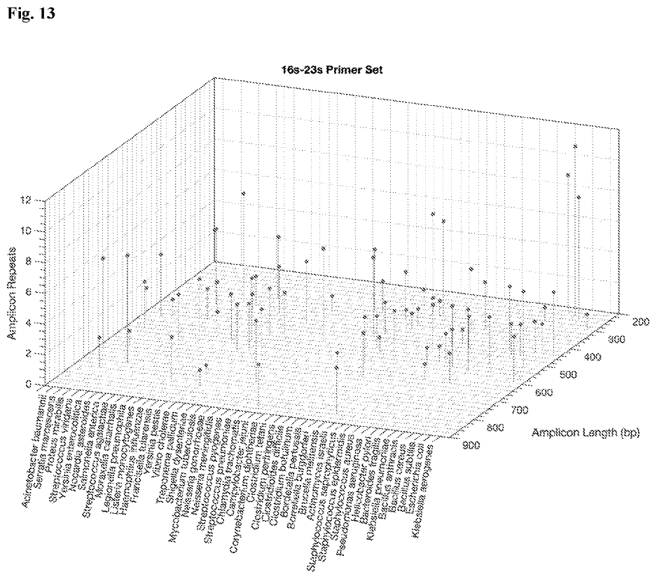

After developing optimized universal primers, predicted amplification profiles of the 16S-23s ITS (FIG. 5) and 23S-5s ITS (FIG. 6) regions, as well as number of ITS repeats (FIG. 7, FIG. 13), using the universal primer sets were generated for the 45 most commonly isolated bacterial pathogen. Predicted amplification profiles of the 16S-23S and 23S-52 ITS regions (FIG. 8), as well as the number of associated ITS repeats (FIG. 11), using the universal primer sets were also generated for a database comprising 188 pathogens.

### Example 2—In Vitro Tests of Bacterial Species

Seven representative bacterial species, including Gram-negatives and Gram-positives, aerobes and anaerobes, and spore formers were tested using the custom 16S-23S universal primer set. (FIG. 9) These results provide unique PCR reactant profiles for each species that closely aligns with the nonoverlapping amplicon profiles generated via bioinformatic simulation (Table 4).

TABLE 4
Comparison of predicted vs observed amplicon
lengths (bp) and number of associated repeats

| | | |
|---|---|---|
| E. coli | 444 (2), 521 (5) | 525 ± 19, 605 ± 19 |
| S. aureus | 456 (2), 551 (2), | 493 ± 9, 582 ± 20, |
| | 638 (1) | 638 ± 35 |
| B. subtilis | 257 (8), 437 (2) | 337 ± 39, 437 ± 25 |
| A. baumannii | 696 (6) | 701 ± 15 |
| P. aeruginosa | 561 (4) | 605 ± 19 |
| K. pneumoniae | 288 (1), 433 (4), | 361 ± 26, 507 ± 20, |
| | 517 (3) | 555 ± 17 |
| C. jejuni | 889 (3) | 925 ± 38 |

*Values are mean ± standard deviation from 3 independent samples

### Example 3—Infectious/Load Concentration for Bacterial Species

The capacity to quantify the corresponding infectious load/concentration for an identified bacterial species using semiquantitative gel analysis and qPCR was tested. (FIG. 2) Aliquots were periodically sampled every 3 cycles during the PCR reaction and analyzed by PAGE. These data were compared to continuous monitoring of the reaction using SYBR Green real-time qPCR. The results confirm the log 2-linear relationship between PCR cycle and concentration, even for these complex (i.e., more than one product) amplicons, and show that the universal ITS primers are of use to determine the concentration of an unknown bacterial sample. Continuous monitoring of the sample every 3 cycles additionally supports characterization of band intensities before the qPCR amplification threshold cycle is reached, from which ITS ratios can be determined.

### Example 4—Sensitivity of Serial Dilution

The sensitivity of the methods, compositions, systems and kits of the present invention were tested using serial 10-fold dilutions of E. coli. (FIG. 3). Under the prescribed conditions, the system detects bacterial DNA at as low of a concentration as 30 μg/uL which corresponds to approximately 250 cells per reaction.

### Example 5—Performance in the Presence of Nontarget DNA

To mimic conditions that encountered in blood, where human DNA can outcompete bacterial DNA by several orders of magnitude, increasingly dilute concentrations of bacterial DNA was mixed with human genomic DNA and amplified.²¹ (FIG. 3) The universal primers do not amplify human genome within a typical number of PCR cycles (i.e., 35 cycles) and produce only very faint bands when significantly over cycled. Of note, the bands produced by the human genome are distinct and can be separated out from the unique band signature produced by bacteria. In addition, the presence of very high concentrations of human DNA does not interfere with the ability to amplify and detect bacterial DNA, even at the sensitivity limit of the system.

### Example 6—Multiple Bacterial Species in a Single Reaction

Given that the system is resilient to off-target DNA, including human DNA, we sought to determine whether the system can reliably detect multiple bacterial species in a single reaction. (FIG. 4) We observed that both E. coli and B. subtilis can be independently identified when present in near equimolar concentrations however amplification of the more dilute species appears to be hindered when present at less than a 1:10 ratio relative to the dominant species.

### Example 7—Clinical Samples

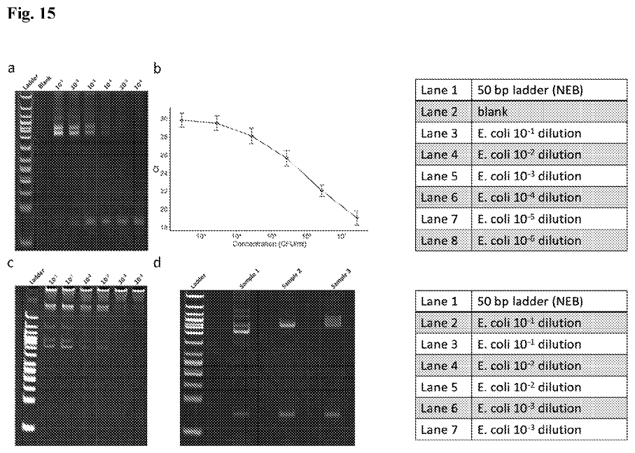

To determine the universal microbial identification system's performance in clinical samples in which potential PCR inhibitors or other matrix constituents may confound analysis, we tested whether bacteria can reliably be detected and identified in 2 of the most commonly collected clinical biofluids i.e., urine and blood. Spiking serial 10× dilutions of E. coli into porcine urine had no effect on the expected PAGE band pattern. (FIG. 15a) In addition, when using the universal primers in conjunction with SYBR green qPCR, the assay was able to reliably detect <10⁵ CFU/ml in urine which is sufficient to diagnose a urinary tract infection (UTI). (FIG. 15b) Similar PAGE results were obtained for serial dilutions of E. coli in whole blood, (FIG. 15c) In keeping with culture results, the methods, compositions, systems and kits of the present invention reliably identify clinically-relevant bacterial pathogens in human UTI samples. (FIG. 13d) For example, sample 1 had a gel pattern consistent with E. coli while samples 2 and 3 had patterns consistent with P. aeruginosa. (Table 5.)

TABLE 5
Clinical urine specimens

| | |
|---|---|
| Sample 1 | E. coli |
| Sample 2 | P. aeruginosa |
| Sample 3 | P. aeruginosa |

### Example 8—Fungal Samples

To establish the efficacy of the universal microbial identification system of the present invention on fungi, universal primers designed against the 18S-5.8S fungal ITS region were tested on varying concentrations of Candida albicans, ranging from 10 to 989 ng/uL. (FIG. 12) Following an overnight culture, the yeast DNA was extracted and amplified via PCR. Candida DNA was then run on a 10% PAGE gel for amplicon signature length determination. Concentrations equaling or above 400 ng/uL of fungal DNA were detectable via gel. Based on simulation data, the expected band size for Candida albicans using the universal primers is 377 bp. Experimental band size was determined to be approximately 370 b showing concordance between in silico and experimental results.

### Example 9—Melt Curve Analysis

High-resolution melt analysis was performed in silico (using uMELT) on six bacteria with overlapping amplicon length signatures in order to show the discriminatory benefit conferred by incorporation of high-resolution melt curve analysis. (FIG. 11a) Amplicons generated by 6 bacteria (G. vaginalis, M. phlei, T. denticola, M. fortuitum, C. jeikeium, and C. jejuni) comprise distinct in silico melt curves despite having similar length signatures. Melt analysis was performed on 6 bacteria (B. subtilis, C. jejuni, E. coli, K. pneumoniae, P. aeruginosa, and S. aureus) (FIG. 11b) using standard melt-curve analysis. Melt analysis requires no additional time to diagnosis and or additional sample because melt curves are characterized immediately after qPCR on the same instrument used for amplification, and simultaneous with electrophoresis.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

### INCORPORATION BY REFERENCE

All publications, published patent documents, and patent applications cited herein are hereby incorporated by reference to the same extent as though each individual publication, published patent document, or patent application was specifically and individually indicated as being incorporated by reference.

### REFERENCES

1. Lagier, J C, Edouard, S, Pagnier, I, Mediannikov, O, Drancourt, M, and Raoult, D (2015). Current and past strategies for bacterial culture in clinical microbiology. Clin Microbiol Rev 28:208-236.
2. Kethireddy, S, Bilgili, B, Sees, A, Kirchner, H L, Ofoma, U R, Light, R B, et al. (2018). Culture-Negative Septic Shock Compared With Culture-Positive Septic Shock: A Retrospective Cohort Study. Crit Care Med 46:506-512.
3. Stewart, E J (2012). Growing unculturable bacteria. J Bacteriol 194:4151-4160.
4. Opota, O, Croxatto, A, Prod'hom, G, and Greub, G (2015). Blood culture-based diagnosis of bacteraemia: state of the art. Clin Microbiol Infect 21:313-322.
5. Peters, R P, van Agtmael, M A, Danner, S A, Savelkoul, P H, and Vandenbroucke-Grauls, C M (2004). New developments in the diagnosis of bloodstream infections. Lancet Infect Dis 4:751-760.
6. Zhang, D, Yang, D, and Makam, A N (2019). Utility of Blood Cultures in Pneumonia. Am J Med 132:1233-1238.
7. Weinstein, M P (2003). Blood culture contamination: persisting problems andpartial progress. J Clin Microbiol 41:2275-2278.
8. Hall, K K, and Lyman, J A (2006). Updated review of blood culture contamination. Clin Microbiol Rev 19:788-802.
9. McGregor, C (2014). Improving time to antibiotics and implementing the “Sepsis 6”. BMJ Qual Improv Rep 2.
10. Seymour, C W, Gesten, F, Prescott, H C, Friedrich, M E, Iwashyna, T J, Phillips, G S, et al. (2017). Time to Treatment and Mortality during Mandated Emergency Care for Sepsis. N Engl J Med 376:2235-2244.
11. CDC (2019). Antibiotic resistance threats in the United States, 2019.
12. Willyard, C (2017). The drug-resistant bacteria that pose the greatest health threats. Nature 543:15.
13. Prestinaci, F, Pezzotti, P, and Pantosti, A (2015). Antimicrobial resistance: a global multifaceted phenomenon. Pathog Glob Health 109:309-318.
14. Chokshi, A, Sifri, Z, Cennimo, D, and Horng, H (2019). Global Contributors to Antibiotic Resistance. J Glob Infect Dis 11:36-42.
15. VanEpps, J S (2018). Reducing exposure to broad-spectrum antibiotics for bloodstream infection. Journal of Laboratory and Precision Medicine 3.
16. Langdon, A, Crook, N, and Dantas, G (2016). The effects of antibiotics on the microbiome throughout development and alternative approaches for therapeutic modulation. Genome Med 8:39.
17. Healthcare Infection Control Practices Advisory Committee. Antibiotic Stewardship Statement for Antibiotic Guidelines—The Recommendations of the Healthcare Infection Control Practices Advisory Committee (HICPAC). 2016.
18. Shi, Y, Wang, G, Lau, H C, and Yu, J (2022). Metagenomic Sequencing for Microbial DNA in Human Samples: Emerging Technological Advances. Int J Mol Sci 23.
19. Tran, A, Alby, K, Kerr, A, Jones, M, and Gilligan, P H (2015). Cost Savings Realized by Implementation of Routine Microbiological Identification by Matrix-Assisted Laser Desorption Ionization-Time of Flight Mass Spectrometry. J Clin Microbiol 53:2473-2479.
20. Stewart, F J, and Cavanaugh, C M (2007). Intragenomic variation and evolution of the internal transcribed spacer of the rRNA operon in bacteria. J Mol Evol 65:44-67.
21. Vitale, D. Comparing the Agilent 2100 Bioanalyzer performance to traditional DNA analysis techniques.
22. Lin, C H, Wang, Y N, and Fu, L M (2012). Integrated microfluidic chip for rapid DNA digestion and time-resolved capillary electrophoresis analysis. Biomicrofluidics 6:12818-1281811.
23. Rowe, T A, and Juthani-Mehta, M (2014). Diagnosis and management of urinary tract infection in older adults. Infect Dis Clin North Am 28:75-89.
24. Gosiewski, T, Ludwig-Galezowska, A H, Huminska, K, Sroka-Oleksiak, A, Radkowski, P, Salamon, D, et al. (2017). Comprehensive detection and identification of bacterial DNA in the blood of patients with sepsis and healthy volunteers using next-generation sequencing method—the observation of DNAemia. Eur J Clin Microbiol Infect Dis 36:329-336.
25. Kainz, P (2000). The PCR plateau phase-towards an understanding of its limitations. Biochim Biophys Acta 1494:23-27.
26. Kais, M, Spindler, C, Kalin, M, Ortqvist, A, and Giske, C G (2006). Quantitative detection of Streptococcus pneumoniae, Haemophilus influenzae, and Moraxella catarrhalis in lower respiratory tract samples by real-time PCR. Diagn Microbiol Infect Dis 55: 169-178.
27. Konig, C, Simmen, H P, and Blaser, J (1998). Bacterial concentrations in pus and infected peritoneal fluid—implications for bactericidal activity of antibiotics. J Antimicrob Chemother 42:227-232.
28. Bergkessel, M, and Guthrie, C (2013). Colony P C R. Methods Enzymol 529:299-309.
29. Franchetti, L, Schumann, D M, Tamm, M, Jahn, K, and Stolz, D (2020). Multiplex bacterial polymerase chain reaction in a cohort of patients with pleural effusion. BMC Infect Dis 20:99.
30. GenMarkDx Blood Culture Identification (BCID) Panels.
31. Chakravorty, S, Helb, D, Burday, M, Connell, N, and Alland, D (2007). A detailed analysis of 16S ribosomal RNA gene segments for the diagnosis of pathogenic bacteria. J Microbiol Methods 69:330-339.
32. Coenye, T, and Vandamme, P (2003). Intragenomic heterogeneity between multiple 16S ribosomal RNA operons in sequenced bacterial genomes. FEMS Microbiol Lett 228:45-49.
33. Vetrovsky, T, and Baldrian, P (2013). The variability of the 16S rRNA gene in bacterial genomes and its consequences for bacterial community analyses. PLOS One 8: e57923.

We claim:

1. A method of identifying multiple different microbes, comprising:
a) contacting nucleic acid from said multiple different microbes with two or more oligonucleotide primers that hybridize to sequence regions of said nucleic acid from said multiple different microbes that are conserved among different microbes, wherein said conserved regions flank variable ribosomal internal transcribed regions (ITS) and conserved genes to produce one or more amplification products;
b) determining the fragment lengths of said one or more amplification products; and
c) identifying said multiple different microbes by comparing said fragment lengths to a database of fragment lengths from a plurality of different microbes.

2. The method of claim 1, wherein said multiple microbes are bacteria and said variable ribosomal internal transcribed regions are 16S-23S and 23S-5S variable ribosomal internal transcribed regions.

3. The method of claim 1, wherein said multiple microbes are fungi and said variable ribosomal internal transcribed regions are 18S-5.8S and 5.8S-28S variable ribosomal internal transcribed regions.

4. The method of claim 1, wherein at least one of said one or more amplification products is amplified by polymerase chain reaction (PCR), quantitative PCR (qPCR) and/or colony PCR.

5. The method of claim 1, wherein high-resolution melt analysis is performed on said one or more amplification products.

6. The method of claim 1, wherein the number of ITS repeats are quantified by qPCR or electrophoresis.

7. The method of claim 1, wherein the fragment length of at least one of said one or more amplification products is determined by gel electrophoresis or capillary electrophoresis.

8. The method of claim 1, wherein at least one of said two or more primers comprises a mixed base and/or an inosine.

9. The method of claim 1, further comprising periodic removal of dominant amplification products followed by replenishment of PCR reagents and serial amplification.

10. The method of claim 1, wherein a concentration of at least one of said one or more amplification products is determined by fluorescent quantification.

11. The method of claim 1, wherein said conserved regions flank one or more variable regions in tRNA synthetase, nirS, rpo, COX1, rbcL, LSU, fus, ileS, lep, leu, pyrG, rps, dna, rnp, rpm, gyr, rec, rpl, and tuf.

12. A system, comprising:
a) two or more amplification primers that hybridize to sequence regions of nucleic acid from multiple different microbes that are conserved among different microbes, wherein said conserved regions flank variable ribosomal internal transcribed regions (ITS) and conserved genes to produce one or more amplification products;
b) one or more PCR amplification reagents;
c) a PCR amplification instrument;
d) a melt curve instrument;
e) one or more capillary gel electrophoresis reagents; and
f) a capillary gel electrophoresis instrument.

13. The system of claim 12, wherein said multiple microbes are bacteria and said variable ribosomal internal transcribed regions are 16S-23S and 23S-5S variable ribosomal internal transcribed regions.

14. The system of claim 12, wherein said multiple microbes are fungi and said variable ribosomal internal transcribed regions are 18S-5.8S and 5.8S-28S variable ribosomal internal transcribed regions.


SEQUENCE LISTING (8 sequences)

SEQ ID NO: 1 - DNA, 21 bp, Synthetic construct
  source 1..21: mol_type = other DNA
  modified_base 21: mod_base = i
aagtcgtaacaaggtarccgn

SEQ ID NO: 2 - DNA, 17 bp, Synthetic construct
  source 1..17: mol_type = other DNA
  modified_base 17: mod_base = i
ygccaaggcatccaccn

SEQ ID NO: 3 - DNA, 17 bp, Synthetic construct
  source 1..17: mol_type = other DNA
  modified_base 17: mod_base = i
gctgaaagcatctaagn

SEQ ID NO: 4 - DNA, 21 bp, Synthetic construct
  source 1..21: mol_type = other DNA
  modified_base 21: mod_base = i
cgtttcacttctgagttcggn

SEQ ID NO: 5 - DNA, 17 bp, Synthetic construct
  source 1..17: mol_type = other DNA
cttgtacwcaccgcccg

SEQ ID NO: 6 - DNA, 16 bp, Synthetic construct
  source 1..16: mol_type = other DNA
ttcgytcgccrctact

SEQ ID NO: 7 - DNA, 21 bp, Synthetic construct
  source 1..21: mol_type = other DNA
  modified_base 21: mod_base = i
gtccctgccctttgtacacan

SEQ ID NO: 8 - DNA, 21 bp, Synthetic construct
  source 1..21: mol_type = other DNA
  modified_base 21: mod_base = i
tttcgctgcgttcttcatcgn